(19) United States
(12) Patent Application Publication
(10) Pub. No.: US 2020/0152328 A1
(43) Pub. Date: May 14, 2020

(54) COGNITIVE ANALYSIS FOR IDENTIFICATION OF SENSORY ISSUES

(71) Applicant: International Business Machines Corporation, Armonk, NY (US)

(72) Inventors: Michael Bender, Rye Brook, NY (US); Gregory J. Boss, Saginaw, MI (US); Rhonda L. Childress, Austin, TX (US); Edward Tyrone Childress, Austin, TX (US)

(21) Appl. No.: 16/186,939

(22) Filed: Nov. 12, 2018

(51) Int. Cl. G16H 50/20; G06N 5/04; G06N 7/00; G06N 99/00; G06F 21/62

(52) U.S. Cl. CPC G16H 50/20; G06N 5/043; G06F 21/6218; G06N 99/005; G06N 7/005

(57) ABSTRACT

A method, computer program product, and a system where a processor(s), obtains a request to be electronically monitored, from a user, via a computing resource, and the request comprises authorization to access one or more data sources utilized by the user or proximate to the user. The processor(s) monitors data sources to obtain data relevant to a user, to generate and train a predictive model to determine a probability that the user is experiencing a sensory issue. The processor(s) trains the model with additional data comprising behavior(s) indicating the sensory issue and contextual factor(s). The processor(s) determines the user is exhibiting, during the time period, the behavior(s) and the processor(s) (deviations from the expected behavior(s)) and determines a context for each incidence of the behavior(s) during the time period. The processor(s) adjusts a portion of the instances to generate an adjusted portion and cognitively analyzes the adjusted portion.

### BACKGROUND

Individuals who suffer from medical issues, such as hearing and sight impairment, may not be cognizant of progressive deterioration, for example, deterioration of hearing and sight. As a results, these individuals may not take measures to enable them to fully appreciate their environments. While symptoms of deterioration may not be readily apparent to those suffering deterioration is health issues, there are various physically observable signs of deterioration, and for example of sight and hearing. For example, for sight and hearing, which are merely two examples of sensory issues, various behaviors that are indicative of some impairment include, but are not limited to, people squinting to read something from a distance, people taking of their glasses off to read items that are close, people asking a speaker to repeat what was just said by that speaker, people rubbing their eyes more often than usual, people changing their positioning, including leaning in, to hear or see something, people answering the wrong question during a conversation, and/or people holding a phone progressively further away from themselves. Because of the progressive nature of the loss of various senses and abilities, including but not limited to, hearing and sight loss, and the slow, yet steady, changes in behaviors that an individual utilizes to compensate for the loss, the individual may not be able to take steps to mitigate the loss, as it is occurring, and may, instead, acclimate to a new normal. The progressive nature of sensory impairment is not limited to sight and hearing and rings true across many senses, including but not limited to: taste, smell, touch, balance and acceleration, temperature, proprioception, pain, time, agency, and/or familiarity. This new normal may create challenges not only for the individual, but also, for others who attempt to interact with this individual. These others may change their behaviors in a way that is detrimental to the individual, sometimes out of frustration. For example, in the example of hearing loss, an individual may be excluded from conversations as others grow tired of repeating questions and receiving incorrect answers.

### SUMMARY

Shortcomings of the prior art are overcome and additional advantages are provided through the provision of a method for identifying sensory issues experienced by users utilizing or proximate to computing device. The method includes, for instance: obtaining, by one or more processors, a request to be electronically monitored, from a user, via a computing resource, wherein the request comprises authorization to access one or more data sources utilized by the user or proximate to the user; continuously monitoring, by the one or more processors, the authorized one or more data sources to obtain data relevant to the user; generating and training, by the one or more processors, a predictive model, wherein the predictive model is utilized by the one or more processors, to determine a probability that the user is experiencing a sensory issue, based on the continuously monitoring, and obtaining additional data, via an Internet connection, from one or more computing resources communicatively coupled to the one or more processors, wherein the additional data comprises one or more behaviors indicating the sensory issue and one or more contextual factors that contribute to the one or more behaviors, wherein the data relevant to the user is utilized by the one or more processors to establish ranges of expected behaviors for the user, when the user is engaged in specific activities, wherein the predictive model comprises the expected behaviors for the user; and cognitively analyzing, by the one or more processors, based on applying the predictive model, a portion of the data obtained by the continuously monitoring during a given time period, to determine that a user is exhibiting the one or more behaviors indicative of the sensory issue, wherein the one or more behaviors represent deviations, during the given time period, from one or more of the established ranges of expected behaviors for the user.

Shortcomings of the prior art are overcome and additional advantages are provided through the provision of a computer program product for identifying sensory issues experienced by users utilizing and/or proximate to computing devices. The computer program product comprises a storage medium readable by a processing circuit and storing instructions for execution by the processing circuit for performing a method. The method includes, for instance: obtaining, by the one or more processors, a request to be electronically monitored, from a user, via a computing resource, wherein the request comprises authorization to access one or more data sources utilized by the user or proximate to the user; continuously monitoring, by the one or more processors, the authorized one or more data sources to obtain data relevant to the user; generating and training, by the one or more processors, a predictive model, wherein the predictive model is utilized by the one or more processors, to determine a probability that the user is experiencing a sensory issue, based on the continuously monitoring, and obtaining additional data, via an Internet connection, from one or more computing resources communicatively coupled to the one or more processors, wherein the additional data comprises one or more behaviors indicating the sensory issue and one or more contextual factors that contribute to the one or more behaviors, wherein the data relevant to the user is utilized by the one or more processors to establish ranges of expected behaviors for the user, when the user is engaged in specific activities, wherein the predictive model comprises the expected behaviors for the user; and cognitively analyzing, by the one or more processors, based on applying the predictive model, a portion of the data obtained by the continuously monitoring during a given time period, to determine that a user is exhibiting the one or more behaviors indicative of the sensory issue, wherein the one or more behaviors represent deviations, during the given time period, from one or more of the established ranges of expected behaviors for the user.

Methods and systems relating to one or more aspects are also described and claimed herein. Further, services relating to one or more aspects are also described and may be claimed herein.

Additional features are realized through the techniques described herein. Other embodiments and aspects are described in detail herein and are considered a part of the claimed aspects.

### BRIEF DESCRIPTION OF THE DRAWINGS

One or more aspects are particularly pointed out and distinctly claimed as examples in the claims at the conclusion of the specification. The foregoing and objects, features, and advantages of one or more aspects are apparent from the following detailed description taken in conjunction with the accompanying drawings in which:

FIG. 1 is a technical environment into which certain aspects of an embodiment of the present invention can be integrated;

FIG. 2 is a workflow illustrating various aspects of an embodiment of the present invention;

FIG. 3 is a workflow illustrating various aspects of an embodiment of the present invention;

FIG. 4 depicts one embodiment of a computing node that can be utilized in a cloud computing environment;

FIG. 5 depicts a cloud computing environment according to an embodiment of the present invention; and

FIG. 6 depicts abstraction model layers according to an embodiment of the present invention.

### DETAILED DESCRIPTION

The accompanying figures, in which like reference numerals refer to identical or functionally similar elements throughout the separate views and which are incorporated in and form a part of the specification, further illustrate the present invention and, together with the detailed description of the invention, serve to explain the principles of the present invention. As understood by one of skill in the art, the accompanying figures are provided for ease of understanding and illustrate aspects of certain embodiments of the present invention. The invention is not limited to the embodiments depicted in the figures.

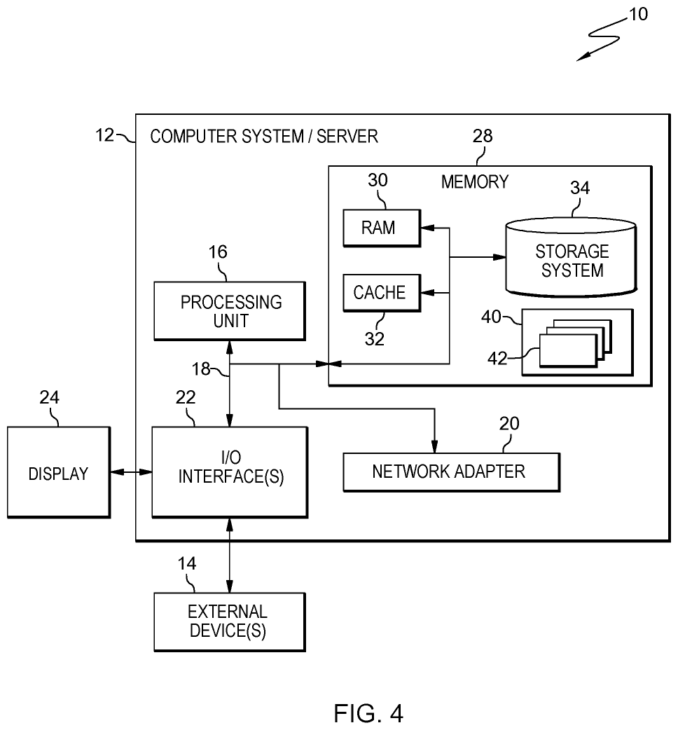

As understood by one of skill in the art, program code, as referred to throughout this application, includes both software and hardware. For example, program code in certain embodiments of the present invention includes fixed function hardware, while other embodiments utilized a software-based implementation of the functionality described. Certain embodiments combine both types of program code. One example of program code, also referred to as one or more programs, is depicted in FIG. 4 as program/utility 40, having a set (at least one) of program modules 42, may be stored in memory 28.

Embodiments of the present invention include a computer-implemented method, a computer program product, and a computing system where program code executing on one or more processors detect and identify sensory issues experienced by a user, including but not limited to visual and audio issues, such that this individual is aware of these issues and can take steps to mitigate them, including at the recommendation of the program code. In some embodiments of the present invention, the program code receives data from sensors proximate to an individual (including in Internet of Things (IoT) devices). The types of sensors can include, but are not limited to, image and sound capture devices (e.g., cameras, microphones), and motion detection devices (e.g., gyroscope). In some embodiments of the present invention, the program code performs a cognitive analysis of the data and based on this cognitive analysis, the program code identifies issues experiences by the individual related to one or more of sight and/or hearing, and based on identifying these issues, the program code adjusts computing devices utilized by the individual to accommodate these issues and/or notifies the individual or others authorized by the individual (e.g., contacts, emergency services) of this issues. In some embodiments of the present invention, the individual registers to utilize the functionality of the program code to monitor the individual and as part of registering, provides permission for the program code to access the individual's IoT or other personal devices. Thus, embodiments of the present invention include program code executing on one or more processor that: 1) captures and analyzes sensor data to identify potential sensory issues experienced by a user, including but not limited to, vision and hearing problems; 2) generates recommendations to the user to seek medical intervention(s); and 3) determines settings for the personal computing devices of the user that mitigate the identified issues and automatically adjusts aspects of personal computing devices utilized by the user to these settings (e.g., resolution, font size, volume, zoom, contrast, etc.).

Over the course of time, individuals may experience changes in their perceptions, related to various senses. With the prior express consent of an individual, embodiments of the present invention can utilize various monitoring of behaviors of the individual in order to identify these issues to the user and to assist the user in mitigating issues earlier than the user notices the issue, unaided by the program code. Visual, olfactory, touch, facial recognition, voice recognition, neuro-cognition (e.g., learning, memory, perception, and problem solving) and/or auditory issues experienced by a user are examples of sensory issues that can be identified by program code in embodiments of the present invention, for which the program code can change personal computing device settings to mitigate or recommend additional assistance, through transmitting a notification. Other senses over which embodiments of the present invention may identify changes in, include, but are not limited to, taste, smell, touch, balance and acceleration, temperature, proprioception, pain, time, agency, and/or familiarity. However, as understood by one of skill in the art, with the prior authorization of the user, aspects of the present invention utilized to monitor user behavior and identify deviations and patterns can be utilized to identify issues with various senses, depending upon the behaviors identified by the program code. For example, in some embodiments of the present invention, the program code can identify user behavioral patterns that indicate changes in sensitivity of touch (e.g., a user making inputs into a personal computing device with increased force). In order to illustrate the functionality and benefits of embodiments of the present invention, rather than to suggest any limitations, in some situation, visual and audio examples are used throughout. As understood by one of skill in the art, these visual and audio examples are offered for illustrative purposes only and do not suggest any limitations on aspects of the invention and their application to tracking behaviors of the user, with the permission of the user, to alert the user to possible sensory issues that user may be experiencing.

Aspects of various embodiments of the present invention are inextricably tied to computing and provide significant advantages over existing approaches to identify visual and hearing issues experienced by a user. First, embodiments of the present invention enable program code executing on one or more processors to exploit the interconnectivity of various systems, as well as utilize various computing-centric data analysis and handling techniques, in order to generate a continuously-updated behavioral baseline model for an individual. The program code applies the model in order to identify deviations in behavior that could be indicative of changes in user behavior over time, which provide indicators of changes in a user's information perception abilities. Both the interconnectivity of the computing systems utilized and the computer-exclusive data processing techniques utilized by the program code enable various aspects of the present invention. Computerized monitoring and analysis techniques are utilized by the program code to analyze user interaction with computing devices, rendering embodiments of the present invention inextricably tied to computing for this reason as well. Second, embodiments of the present invention provide advantages over existing techniques of monitoring changes in sensory behaviors if users, including eyesight and hearing monitoring techniques, because rather the require complex devices to diagnose an issue, at the time an individual is already seeking assistance for this perceived issue (e.g., noting an eyesight problem during an eye exam), through cognitive analysis, embodiments of the present invention progressively analyze behaviors of a user and both identify and mitigate issues at times when a user may not be aware of the existence of issues. Rather than relying upon expensive medical equipment, in embodiments of the present invention, sensors, some of which are included in various computing devices utilized by the user, with the permission of the user, identify issues based on observed behaviors of the user. While existing techniques of identifying sensory issues are constrained because each sense requires the use of specialized equipment, specifically for use in identifying issues with that sense, embodiments of the present invention can be utilized across a variety of senses. For example, one existing approach to vision issues is to diagnose those issue through the use of an eye chart while another is not use a specialized piece of medical equipment. In both these approaches, unlike embodiments of the present invention, the approach does not identify issues by monitoring user behavior utilizing sensors nor make automatic adjustments to devices to mitigate identified issues. Another existing approach changes the volume on a specific set of speakers based on a hearing test conducted using those speakers. Again, this approach does not monitor user behavior, such as movements, as will be explained below, to identify auditory ideas and unlike aspects of embodiments of the present invention, in this approach, any modifications are limited to a specific speaker. Thus, existing techniques are limited to utilizing a specific approach for a specific sense, while embodiments of the present invention are applicable across a myriad of senses.

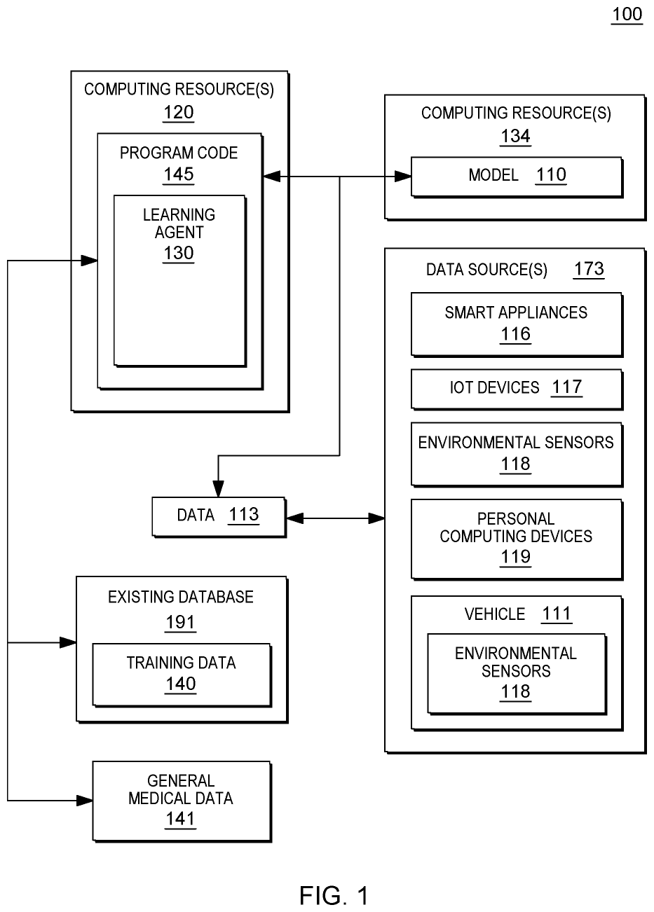

FIG. 1 is a technical environment 100 into which aspects of some embodiments of the present invention can be integrated and includes one or more computing resources 120 which execute program code 145 that include a cognitive learning agent 130 that identifies and analyzes trends in data collected by the program code 145. The one or more computing resources 120 execute program code 145 that generates or updates a model 110, based on machine learning (e.g., via a cognitive and/or learning agent 130), and utilizes the model 100 to identify a behavioral patterns that indicate sensory issues, experienced by a user, who has opted into monitoring and data acquisition by the program code 145. For illustrative purposes only, the model 110 is depicted in FIG. 1 as being housed on a separate computing resource 134 from the one or more computing resources 120 that execute the program code 145. This is a non-limiting example of an implementation, as the program code 145 and the model 110 can also share a computing resource. Likewise, in the illustrated implementation, the program code 145 is illustrated as comprising the learning agent 130. However, various modules of the program code 145 can be executed on varied resources in various embodiments of the present invention, thus, the learning agent 130 and the program code 145 can be separate modules.

In embodiments of the present invention, program code 145 utilizes various data 113 from various sensors, cameras (or other image capture devices), biometric feedback, manual inputs, to monitor activity of a user interacting with the sensors and/or proximate to the sensors. In order to respect the privacy of the user and to ensure the security of aspects of the present invention, in embodiments of the present invention, a user acquiesces to the use of various data 113 in monitoring, including, in some embodiments of the present invention, expressly registered devices to provide the various data 113 through a graphical user interface (GUI). This data 113, chronicling the activity of the user, can be provided from various data sources 173, including but not limited to, smart appliances 116 (televisions, kitchen devices, etc.), personal computing devices 119, utilized by an individual and proximate to an individual, Internet of Things (IoT) devices 117, and environmental sensors 118 in various environments, including sensors inside a vehicle 111 being operated by the individual. Certain of the one or more data sources 173 can be publicly available and the data can include publicly available IoT data from environmental sensors 118, including cameras and microphones in physical settings (e.g., classroom, conference room, retail location, etc.). In embodiments of the present invention, program code utilizing data that is not publicly accessible will obtain permission from a user before utilizing private data, including but not limited to, data with personally identifiable information. In embodiments of the present invention where the program code utilizes publicly available data, the program code can alert a user to the data being accessed by the program code.

The one or more data sources 173, including both the publicly available sources to which the program code alerts the user to the utilization, and the private data sources to which the user has given the program code permission to access, effectively comprise a continuous monitoring system within the environment 100. As noted above, the data 113 can include data collected from sensors and devices with sensors, including smart devices 116 (which can be IoT devices), IoT devices 117, environmental sensors 118, and/or personal computing devices 119. In some embodiments of the present invention, the data 113 includes biometric and/or physiological data from continuous monitoring and includes, but is not limited to, cardiovascular measures (e.g., heart rate, blood pressure, blood oxygen saturation, and respiration), body positioning and movement data (e.g., rest versus activity data), body temperature, and environmental conditions of the environment of the patient (e.g., ambient light and/or noise).

As understood by one of skill in the art, the Internet of Things (IoT) is a system of interrelated computing devices, mechanical and digital machines, objects, animals and/or people that are provided with unique identifiers and the ability to transfer data over a network, without requiring human-to-human or human-to-computer interaction. These communications are enabled by smart sensors, which include, but are not limited to, both active and passive radio-frequency identification (RFID) tags, which utilize electromagnetic fields to identify automatically and to track tags attached to objects and/or associated with objects and people. Smart sensors, such as RFID tags, can track environmental factors related to an object, including but not limited to, temperature and humidity. The smart sensors can be utilized to measure temperature, humidity, vibrations, motion, light, pressure and/or altitude. IoT devices 117, in general, also include individual activity and fitness trackers, which include (wearable) devices or applications that include smart sensors for monitoring and tracking fitness-related metrics such as distance walked or run, calorie consumption, and in some cases heartbeat and quality of sleep and include smartwatches that are synced to a computer or smartphone for long-term data tracking. In some embodiments of the present invention, the program code 145 executed by the one or more computing resources 120, with the permission of the user, utilizes IoT devices 117, such as personal fitness trackers and other types of motion trackers, both to establish a baseline (e.g., generate and update a model 110 through machine learning optionally via a learning agent 130) for a user when engaged in a specific activity (e.g., the distance a user sits from a monitor when reading text) and to determine whether the user, who is engaged in the specific activity, is deviating from that activity, based on progressive changes in behavior (e.g., sitting progressively closer to the monitor, when reading, over time). IoT devices also include Smart home devices (noted separately in FIG. 1, for illustrative purposes only, as smart appliances 116), digital assistants, and home entertainment devices, which comprise examples of environmental sensors 118. Because the smart sensors in IoT devices 117 carry unique identifiers, a computing system that communicates with a given sensor can identify the source of the information. Within the IoT, various devices can communicate with each other and can access data from sources available over various communication networks, including the Internet. Thus, the program code 145 in some embodiments of the present invention utilizes data obtained from various IoT devices 117 to generate or update the model 110 utilized by the program code 145 to identify changes in behaviors of a user, over time, which indicate that the user is experiencing sensory perception issues. In some embodiments of the present invention, the program code identifies IoT devices 117 utilized by a user based on the user registering those devices and enabling the program code to access these IoT devices 117. The program code provides a GUI for registration of the IoT devices 117 and monitors the IoT devices 117 only after obtaining permission from the user, through the GUI.

The program code 145 updates the model 110 in real-time, upon receipt of the data 113, including sensor data that deviates from the model 110. Program code 145 of the learning agent 130 utilizes this data 113 to continuously learn and updates the patterns that form the model 110.

As aforementioned, in embodiments of the present invention, the program code 145 executing on the one or more computing resources 120 determines that a user is exhibiting behaviors that deviate from previously established (modeled) behavioral patterns. In addition to utilizing sensor data from various data sources 173

The program code 145 can also receive this data from other types of computing devices, including image capture devices proximate to the user, including (with proper security permissions) embedded in the personal computing devices 119.

In some embodiments of the present invention, the program code 145 determines that a given behavior is indicative of a sensory issue based on utilizing data to train the model 110, which can include training data 140. In some embodiments of the present invention, when the program code 145 initializes the model 110, the program code 145 obtains training data 140 that the program code 145 can utilize to improve pattern detection and prediction (generating and updating the model 110). For example, the training data 140 may indicate specific behaviors that are indicative of sensory issues, based, for example, on these behaviors being progressive and representing trends/patterns over time. Below are some non-limiting examples of trends in the training data 140 that the program code 145 can utilize to initialize the model 110: 1) an individual puts on reading glasses while reading a monitor; 2) an individual squints and/or leans forward when viewing content on a display; 3) an individual interacting with a voice-activated interface repeats requests or responds in a manner that does not indicate comprehension of a requested response from the device; 4) an individual engaged in a conversation via a mobile phone requests repetitions of phrases by another individual engaged in the conversation; 5) an individual changes position to be closer to the source of sound that the individual is attempting to hear; 6) a person provides responses that are not responsive to a question posed; 7) a user depresses a button multiple times; and/or 8) a user does not respond to haptic feedback, etc.

In some embodiments of the present invention, the training data 140 also includes environmental factors that can generate false positives, meaning that an individual is correctly observed by the program code 145 to behave in a manner that, based on the model 110, would indicate a sensory issue, however, the behavior alone is not conclusive because of mitigating factors. Thus, the training data 140 can include general information about possible disturbances such that the model 110 can be trained, by the learning agent 130, to determine, when a given mitigating factor exists, the probability that that mitigating factor impacted the behavior of a user, such that the behavioral trends observed in the data 113 are not indicative of an issue. Mitigating factors can include, but are not limited to: 1) background noise; 2) volume of audio; 3) activities being performed by the user contemporaneously with the activity in which the user is perceived, by the program code 145, to be experiencing issues (e.g., browsing web, typing in another application); 4) graphical user interface (GUI) settings (e.g., font, font size, font color, colors displayed, contrast of colors displayed with each other); 5) ambient light; 6) activities being performed proximate to the user contemporaneously with the activity in which the user is perceived, by the program code 145, to be experiencing issues; 7) ambient noise; 8) quality of media (output) the user is interacting with (e.g., sound, interference, noise, color, clarity); and/or 9) environmental factors. In embodiments of the present invention, the program code 145 can utilize training data 140 that defines thresholds for various mitigating factors, such that the program code 145 can: 1) obtain data 113 from the data sources 173, 2) identify mitigating factors, based on the model 110, that can negatively impact sensory perception of the user monitored to generate the data 113, and 3) determine whether the mitigating factors rise to a threshold where a pre-determined probability exists that the behaviors indicated in the data 133, as identified by the program code 145 applying the model 110, are a result of the mitigating factors, rather than a sensory issue.

The source for the training data 140 can be one or more existing databases 191 or data sources. The existing databases 191 can be publicly available or private (permissioned) systems, that include medical information related to what symptoms and behaviors indicate various sensory issues and what environmental factors can influence these behaviors. In the case of medical information, in embodiments of the present invention, the program code can utilize databases that do not include any personally identifiable information or access only medical data of individuals from which the program code has received express permission. In some embodiments of the present invention, the program code utilizes general medical data 141 as well as another source of training data for the initialization of the model 110 by the program code 145.

In some embodiments of the present invention, the program code 145 also accesses general medical data 141 in order to correlate a behavior of an individual with a sensory issues experienced by that individual. For example, in some embodiments of the present invention, the program code 145 tracks behaviors of an individual (e.g., movement, activities, etc.). The program code 145 continuously monitors behaviors of the individual and determines, based on baseline behavior patterns of the individual, established by the program code 145 and based on receiving data 113 from sensors inside a vehicle 111, the individual is driving in a manner inconsistent with the individual's (safe) driving patterns and is wearing glasses (an observed by an image captured device among the environment sensors 118 in the vehicle). In this example, the program code 145 determines that an individual has begun squinting while operating the vehicle 111 and has moved the seat on the driver side progressively closer to the windshield. Given that the program code 145 has previously learned, via the learning agent 130, patterns and/or known behaviors of the individual, including the placement of the individual's seat and the position of the individual while driving, the program code 145 accesses the general medical data 141 (which can be a specialized database requiring permissions and/or a resource that is publicly available via an Internet connection) and determines, that the individual is experiencing a possible degradation of the individual's eyesight. The program code 145 updates the model 110 to indicate the findings and can also can alert the individual of the issue. As noted above, this eyesight example is utilized for illustrative purposes only as various embodiments of the present invention can monitor various behaviors that indicate possible changes to or issues with additional senses of a user.

As discussed above, the program code 145 executing on one or more computing resources 120 applies machine learning algorithms to generate and train algorithms to generate a model 110 the program code 145 utilizes to identify sensory issues experienced by individuals within the environment 100 (in this example). Based on identifying the issues, the program code can notify the user and/or made an adjustment to a computing device within the environment 100 to mitigate the issue. In the aforementioned initialization stage, the program code 145 trains these algorithms, based on patterns for a given individual as well as known behaviors indicated in training data 140 and/or general medical data 141.

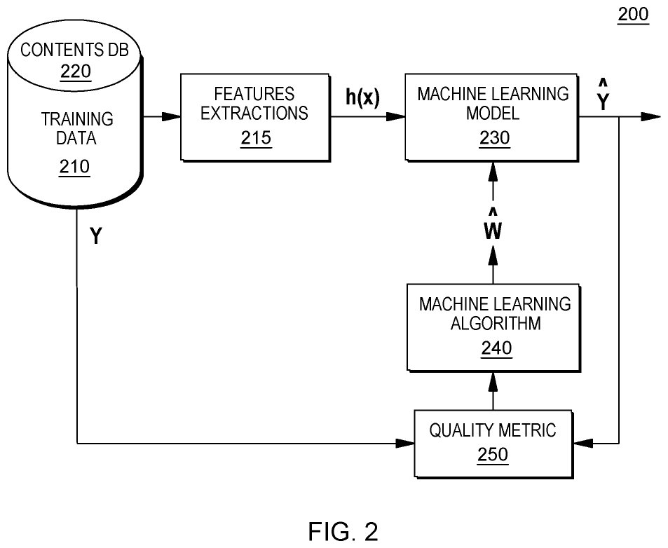

FIG. 2 is an example of a machine learning training system 200 that can be utilized to perform cognitive analyses of various inputs, including the general initialization data, the data 113, and optionally and the training data 140. In addition to what is referred to as the training data 140 (i.e., standard data regarding physical signs of sensory issues and mitigating environmental factors) data utilized to train the model 110 in embodiments of the present invention can also include historical data that is personalized to the individual, including but not limited to the data 113 collected from the data sources 173, which can include, but is not limited to, with the permission of the user, physiological data including cardiovascular measures such as heart rate, blood pressure, blood oxygen saturation, respiration, body movement, body position, motion, and/or temperature. The environmental data that is contemporaneous to the individual data is also collected by the sensors and included in the data 113 and can be used to train the model. This data can include ambient light and noise readings.

FIG. 2 illustrates the machine learning performed by the program code 110 (FIG. 1), via the learning agent 130 (FIG. 1), to generate and continuously updated a model 110 (FIG. 1), in some embodiments of the present invention. Referring to FIG. 2, the program code in embodiments of the present invention performs a cognitive analysis to generate data structures, including algorithms utilized by the program code to identify behavioral patterns of various individuals that indicate a likelihood that the individuals are experiencing sensory issues. Machine learning (ML) solves problems that cannot be solved with numerical means alone. In this ML-based example, program code extracts various features/attributes from training data 240 (e.g., historical data collected from various data sources relevant to the individual and general data), which may be resident in one or more databases 220 comprising individual-related data and general data. The features are utilized to develop a predictor function, h(x), also referred to as a hypothesis, which the program code utilizes as a machine learning model 230. In identifying behavioral patterns that indicate sensory issues as well as environmental factors that can be mitigating factors in the training data 110, the program code can utilize various techniques including, but not limited to, mutual information, which is an example of a method that can be utilized to identify features in an embodiment of the present invention. Further embodiments of the present invention utilize varying techniques to select features (elements, patterns, attributes, etc.), including but not limited to, diffusion mapping, principal component analysis, recursive feature elimination (a brute force approach to selecting features), and/or a Random Forest, to select the attributes related to behaviors exhibited by individuals experiencing sensory issues and environmental factors that may impact these behaviors. The program code may utilize a machine learning algorithm 140 to train the machine learning model 130 (e.g., the algorithms utilized by the program code), including providing weights for the conclusions, so that the program code can train the predictor functions that comprise the machine learning model 130. The conclusions may be evaluated by a quality metric 150. By selecting a diverse set of training data 110, the program code trains the machine learning model 130 to identify and weight various attributes (e.g., features, patterns) that correlate to various behaviors exhibited by a user by an individual, the environmental factors that impact this behavior, and determine, within a discernable probability, if the behavior, in view of the factors, indicates a departure indicative of an issue.

Returning to FIG. 1, the model 110 generated by the program code 145 can be self-learning, as the program code 145 updates the model 110 based on passive feedback received from the data 113, related to monitoring the individual. For example, when the program code 145 determines that an individual regularly sits a distance from a given personal computing device 119 that would indicate an issue with the user's eyesight, but the user displays no similar behavior when utilizing other personal computing devices 119, the program code 145 utilizes a learning agent 130 to update the model 110 to reflect this behavior to improve identification of possible sensory issues in the future. Program code 145 comprising a learning agent 130 cognitively analyzes the data deviating from the modeled expectations and adjusts the model 110 in order to increase the accuracy of the model, moving forward.

In some embodiments of the present invention, program code 145 executing on one or more computing resources 120, utilizes existing cognitive analysis tools or agents to tune the model 110, based on data obtained from the various data sources, including the data 113. Embodiments of the present invention can utilize a variety of existing cognitive agents as well as existing APIs to tune the model 110. Some embodiments of the present invention utilize IBM Watson® as the learning agent 130 (i.e., cognitive agent). IBM Watson® is a product of International Business Machines Corporation. IBM Watson® is a registered trademark of International Business Machines Corporation, Armonk, N.Y., US. IBM Watson® is a non-limiting example of a cognitive agent that can be utilized in embodiments of the present invention and is discussed for illustrative purposes, only, and not to imply, implicitly or explicitly, any limitations regarding cognitive agents that can comprise aspects of embodiments of the present invention.

In some embodiments of the present invention that utilize IBM Watson® as a cognitive agent, the program code 145 interfaces with IBM Watson® APIs to perform a cognitive analysis of obtained data, in some embodiments of the present invention, the program code 145 interfaces with the application programming interfaces (APIs) that are part of a known cognitive agent, such as the IBM Watson® Application Program Interface (API), a product of International Business Machines Corporation, to determine behavioral patterns in the data 113 indicative of sensory issues and to update the model 110, accordingly. Embodiments of the present invention that utilize IBM Watson® may utilize APIs that are not part of IBM Watson® to accomplish these aspects.

In some embodiments of the present invention that utilize IBM Watson®, the program code 145 trains aspects of the IBM Watson® Application Program Interface (API) to learn the relationships between behaviors of individuals monitored by the sensors 113 progressive sensory impairment (e.g., eyesight issues, hearing issues) experienced by the individuals. For example, in some embodiments of the present invention, the program code 145 can determine that a given individual is experiencing a hearing problem, based on incoherent responses, from the individual, to vocal outputs to the individual from a personal computing device 219. In embodiments of the present invention that do not utilize IBM Watson®, the program code 145 trains aspects of various APIs to learn the relationships between behaviors of individuals monitored by the sensors 113 progressive sensory impairment (e.g., eyesight issues, hearing issues) experienced by the individuals.

Although not all embodiments of the present invention utilize an existing cognitive agent, utilizing an existing cognitive agent, such as IBM Watson®, or similar cognitive agents with APIs that can process various types of data, can expand the type of data that the program code 145 can integrate into the model 110. For example, data 213 from various sources can include documentary, visual, and audio data, which the program code 145 can process, based on its utilization of IBM Watson® or another cognitive agent with this capability. Specifically, in some embodiments of the present invention, certain of the APIs of the IBM Watson® API comprise a cognitive agent (e.g., learning agent 130) that includes one or more programs, including, but are not limited to, natural language classifiers, Retrieve and Rank (i.e., a service available through the IBM Watson® Developer Cloud that can surface the most relevant information from a collection of documents), concepts/visual insights, trade off analytics, document conversion, and/or relationship extraction. In an embodiment of the present invention, one or more programs analyze the data obtained by the program code 145 across various sources utilizing one or more of a natural language classifier, retrieve and rank APIs, and trade off analytics APIs. The IBM Watson® Application Program Interface (API) can also provide audio related API services, in the event that the collected data includes audio, which can be utilized by the program code 145, including but not limited to speech recognition, natural language processing, text to speech capabilities, and/or translation. Various other APIs and third party solutions outside of IBM Watson® can also provide this functionality in embodiments of the present invention.

The program code 145 can provide information to individuals regarding sensory issues identified by the program code 145 that individual may be experiencing (e.g., eyesight loss, hearing loss) as varying values. In some embodiments of the present invention, the program code 145 calculates a binary value for the individual, which represents whether a given substance is predicted to effect a given individual during a given time period. In other embodiments of the present invention, the program code 145 provides the user with an indicator of one or more of: 1) a determination that the individual is experiencing a sensory issue; and/or 2) a confidence level related to the determination. As discussed above, in embodiments of the present invention, should the individual's behavior and/or other monitored values deviate from the model 110 determinations, based on continuously monitoring the individual (e.g., utilizing IoT devices 117 and other computing devices including environmental and/or personal sensors), the program code 145 can immediately update the model 120 and/or, in some embodiments of the present invention, alert the individual and/or other users designated by the individual to be alerted. For example, in some embodiments of the present invention, alerts can be sent to medical personnel treating the individual.

In some embodiments of the present invention, the program code 145 utilizes a neural network to analyze collected data relevant to an individual to generate the model 120 utilized to determine if the individual is experiencing any sensory issues and/or changes in senses over time. Neural networks are a biologically-inspired programming paradigm which enable a computer to learn from observational data, in this case, one or more of the data 113 from various data sources 173, the training data 140, and the general medical data 141. This learning is referred to as deep learning, which is a set of techniques for learning in neural networks. Neural networks, including modular neural networks, are capable of pattern (e.g., state) recognition with speed, accuracy, and efficiency, in situations where data sets are multiple and expansive, including across a distributed network, including but not limited to, cloud computing systems. Modern neural networks are non-linear statistical data modeling tools. They are usually used to model complex relationships between inputs and outputs or to identify patterns (e.g., states) in data (i.e., neural networks are non-linear statistical data modeling or decision making tools). In general, program code 145 utilizing neural networks can model complex relationships between inputs and outputs and identify patterns in data. Because of the speed and efficiency of neural networks, especially when parsing multiple complex data sets, neural networks and deep learning provide solutions to many problems in multiple source processing, which the program code 145 in embodiments of the present invention accomplishes when obtaining data and generating a model for determining if a user is experiencing an issue with one or more or the user's senses (e.g., eyesight loss, hearing loss, etc.).

Some embodiments of the present invention may utilize a neural network (NN) to predict future states (e.g., future sensory issues and/or the progression of an identified issue) of a given individual. Utilizing the neural network, the program code 145 can predict the likelihood an individual experiences, for example, hearing loss, eyesight issues, etc., at a subsequent time. The program code 145 obtains (or derives) data related to the individual from various sources to generate an array of values (possible behaviors/sensory issues) to input into input neurons of the NN. Responsive to these inputs, the output neurons of the NN produce an array that includes the predicted issues during predicted time periods. The program code 145 can automatically transmit notifications related to the predicted side effects based on the perceived validity.

In some embodiments of the present invention, a neuromorphic processor or trained neuromorphic chip can be incorporated into the computing resources executing the program code 145. One example of a trained neuromorphic chip that is utilized in an embodiment of the present invention is the IBM® TrueNorth chip, produced by International Business Machines Corporation. IBM® is a registered trademark of International Business Machines Corporation, Armonk, N.Y., U.S.A. Other names used herein may be registered trademarks, trademarks or product names of International Business Machines Corporation or other companies.

The IBM® TrueNorth chip, also referred to as TrueNorth, is a neuromorphic complementary metal-oxide-semiconductor (CMOS) chip. TrueNorth includes a many core network on a chip design (e.g., 4096 cores), each one simulating programmable silicon “neurons” (e.g., 256 programs) for a total of just over a million neurons. In turn, each neuron has 256 programmable synapses that convey the signals between them. Hence, the total number of programmable synapses is just over 268 million (2\hat{ }28). Memory, computation, and communication are handled in each of the 4096 neurosynaptic cores, so TrueNorth circumvents the von-Neumann-architecture bottlenecks and is very energy-efficient.

Below are some non-limiting examples of the functionality of various aspects of some embodiments of the present invention can be utilized in various situations. To illustrate these examples, reference is made to the environment 100 of FIG. 1. The use of the environment 100 of FIG. 1 is not meant to impose any limitations, but merely to provide an illustration for various aspects. For example, a user sits in front of a laptop, which is a personal computing device 119. The laptop camera, a sensor of the laptop, captures images of the user's face. The program code 145 obtains the image data 113 and analyzes the data 113, determining (utilizing the model 110) that the user removes his or her glasses and squints when the font size on the screen of the laptop is less than a twelve point font. In some embodiments of the present invention, the program code automatically changes the zoom on the graphical user interface (GUI) displayed on the laptop such that all fonts are rendered as no smaller than twelve point. In some embodiments of the present invention, the program code 145 notifies the user that he is she may wish to utilize a larger monitor. In another example, program code 145, via data 113 from a laptop camera (e.g., personal computing device 119), and a gyroscope in a user's phone (e.g., personal computing device 119), determines that a user is squinting as well as leaning into displays on a more frequent basis, over time. Based on applying the model 110, the program code determines that this behavioral patterns indicates vision issues and notifies the user that he or she should consider visiting a medical professional, based on the progressive nature of the behavior identified. In another example, a sound input on a mobile phone (e.g., personal computing device 119) provides data to the program code 113, which the program code 145 determines, through analysis, includes conversations where the user asks the second party to repeat statements and also responds with questions or statements that indicate the failure to hear what was said by the other party (e.g., requesting repetition, answering the wrong question, etc.). The program code 145 determines that this behavior is increasing over time and also, that the conversations in which this behavior is occurring are not distorted by background noise. As the program code 145 tracks this behavior over time, it transmits a recommendation to the user to seek medical assistance with a potential hearing issue.

As explained above, in certain situations, the program code 145 can identify a behavior that would indicate as issue, based on the model 110. However, the probability of the issue is decreased (sometimes below as acceptable threshold) based on external factors. For example, the program code 145 can determine, based on obtaining data from a laptop camera (e.g., personal computing device 119) that an individual is squinting while looking at a display in a particular geographic location (as obtained from the location services in the mobile phone e.g., personal computing device 119, of the user). However, the program code 145 continuously monitors the user on subsequent visits to the location and does not observe the same behavioral pattern. Thus, the program code takes no further action. In another example, a sound input on a mobile phone (e.g., personal computing device 119) provides data to the program code 113, which the program code 145 determines, through analysis, includes conversations where the user asks the second party to repeat statements and also responds with questions or statements that indicate the failure to hear what was said by the other party (e.g., requesting repetition, answering the wrong question, etc.). The program code 145 determines that this behavior is persists over time and also, but that the conversations in which this behavior is occurring are distorted by background noise and/or the volume settings are lower than usual on the phone. Based on these mitigating factors, the program code 145 takes no further action.

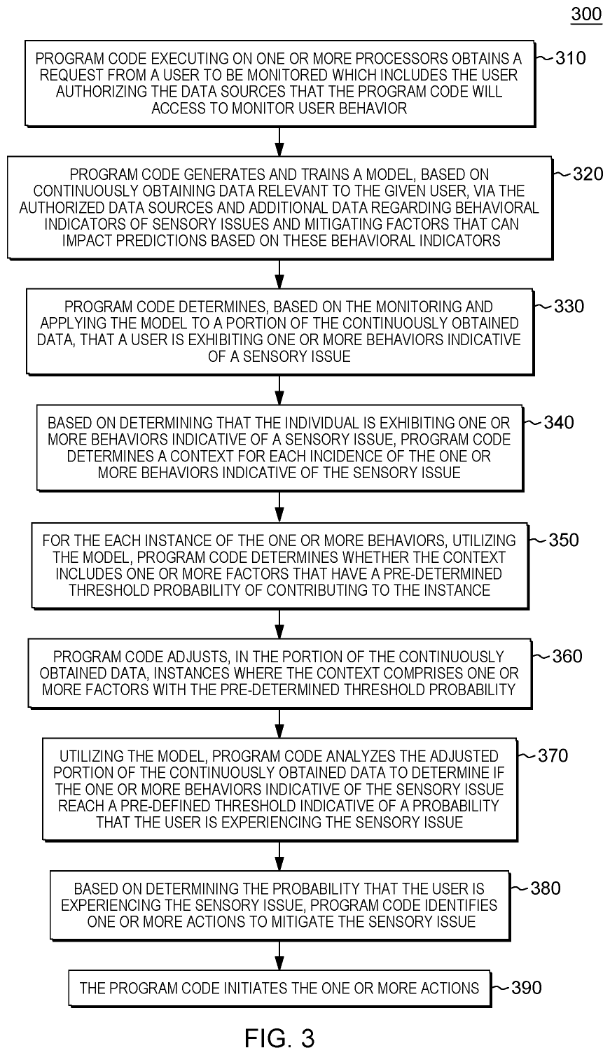

FIG. 3 is a workflow 300 that illustrates certain aspects of some embodiments of the present invention. In some embodiments of the present invention, program code executing on one or more processors obtains a request from a user to be monitored which includes the user authorizing the data sources that the program code will access to monitor user behavior (310). Authorizing the data sources can include: 1) providing permission and credential information for the program code to access one or more of the individual's computing devices (IoT devices, personal computing devices, phones, etc.); and 2) providing permission (or declining to provide permission) for the program code to access publicly available IoT data (e.g., cameras, microphones, etc.) in monitoring user behavior. The request can also include demographic information about the user (e.g., name, address, and age), which the program code can utilize in its analysis. In order to avoid unnecessary alerts, the user can also describe any known sensory problems. The user can register through a graphical user interface (GUI) on a computing resource. Both this subscription procedure and alerts and automatic changes to settings provided by the program code conform to best privacy and security practices. For example, alerts provided by the program code, to which a user subscribes, in some embodiments of the present invention, do not include any personally identifiable information, but, rather, indicate probabilities that an individual is experiencing certain sight or hearing-related issues. This indication can be provided by the program code in a user-friendly manner, through a graphical user interface on a client computing device.

As understood by one of skill in the art, the program code, through training and iterative processing, can establish baseline values that represent behavioral patterns for a given individual. The program code can cognitively analyze the data to identify these patterns and integrate the patterns into the predictive model. Certain values obtained by the program code can deviate outside of expected ranges from the baseline, but as the overall activity or behavioral patterns of the individual changes, the baseline value can also change. In embodiments of the present invention, the program code can obtain updated data describing the movement or habits of the individual when the individual is engaged in a given activity (e.g., reading content on a computing interface, talking on the phone, participating in a meeting, etc.) and update various baselines that comprise the model based on the changes. The program code can update baselines based on threshold changes (changes of a certain degree and/or of a certain quantity).

Returning to FIG. 3, the program code generates and trains a model, based on obtaining data relevant to the given user, via the authorized data sources. As aforementioned, a user has previously registered to be monitored and to provide data to the program code. Hence, the data sources are noted as being authorized (by the user). Program code generates and trains a model, based on continuously obtaining data relevant to the given user, via the authorized data sources and additional data regarding behavioral indicators of sensory issues and mitigating factors that can impact predictions based on these behavioral indicators (320). Based on obtaining data from the authorized sources, the program code determines a baseline for user activity and generates the model, based on the baseline. Thus, the program code can utilize the model to determine when behaviors of a user deviate from the baseline and that these deviations are indicative of (progressive) sensory issues. As part of generating the model, the program code trains or initializes the model, as discussed in FIGS. 1-2.

The program code can initialize the model based on data related to the overall activity of the user, as well as, in some embodiments of the present invention, publicly available data regarding behavioral indicators of sensory issues and mitigating factors that can impact predictions based on these behavioral indicators. As depicted in FIG. 1, the program code can utilize general medical data 141 and the training data 140 to train the model 110. By utilizing this general medical data 141 and the training data 140, the program code can improve the pattern detection and learning utilized by the program code to generate the prediction model. Thus, while the data from the authorized data sources enable the program code to establish a baseline for the user, the publicly available data regarding behavioral indicators of sensory issues and mitigating factors that can impact predictions based on these behavioral indicators enable the program code to correlate behavioral patterns it may identify, through future monitoring, with specific sensory issues. Additionally, the program code can weigh its assessment that a sensory issue exists based on considering mitigating factors present in the data from the authorized data sources.

The program code determines, based on the monitoring and applying the model, a portion of the continuously obtained data, that a user is exhibiting one or more behaviors indicative of a sensory issue (330). As explained in FIG. 1, the program code can make this determination from receiving data 113 from various sources. In analyzing the data with the assistance of the model, program code in various embodiments of the present invention: 1) analyzes obtained audio data to locate phrases that would indicate hearing difficulty; 2) analyzes obtained audio data to locate phrases that indicate repetition of questions as a result of a user answering a different question than that posed; and 3) analyzes visual data to identify known behaviors that indicate perception issues (e.g., removing glasses, rubbing eyes, squinting, leaning in to an audio source or video/image display, turning to orient ears toward an audio source, changing distance between user and audio of video/image source, etc.). As explained above, the program code can utilize an existing cognitive agent (e.g., IBM Watson®) and APIs associated with the agent to perform these analyses of data obtained from the various data sources. Given that the program code continuously obtains the data, the program code in some embodiments of the present invention portions the data in accordance with pre-defined windows of time (e.g., hours, weeks, months, quarters, years, etc.), in order to analyze changes in behavior of a user, progressively, over those windows of time.

Based on determining that the individual is exhibiting one or more behaviors indicative of a sensory issue, the program code determines a context for each incidence or occurrence of the one or more behaviors indicative of the sensory issue (340). In determining the context, the program code identifies, utilizing the model, in the data from the one or more data sources, environmental factors contemporaneous with the behaviors (e.g., background noises, verbal volumes, additional activities simultaneously performed by the user, font size/color, ambient light, etc.). Context includes data gathered by the sensors contemporaneously with the behaviors that indicate a sensory issue. For each instance of the one or more behaviors, utilizing the model, the program code determines whether the context includes one or more factors that have a pre-determined threshold probability of contributing to the instance (350). For example, the program code can determine that a user squinted at a laptop screen at a given time, but the sensor data at this time also indicates that the lighting in the space where the user was situated changed at this time such that the light level was less than average for that space. Thus, the program code (e.g., utilizing a cognitive) can determine that the lighting was a factor in the squinting and therefore, the squinting at that time does not conclusively indicate a vision issue.

The program code adjusts, in the portion of the continuously obtained data, instances where the context comprises one or more factors with the pre-determined threshold probability (360). Utilizing the model, the program code analyzes the adjusted portion of the continuously obtained data to determine if the one or more behaviors indicative of the sensory issue reach a pre-defined threshold indicative of a probability that the user is experiencing the sensory issue (370). To adjust the data, in some embodiments of the present invention, the program code eliminates instances of the one or more behaviors indicative of the sensory issue where the one or more factors have the pre-determined threshold probability before determining whether a user is experiencing the sensory issue. In other embodiments of the present invention, the program code weights instances of the behavior and can assign a lighter weight when contextual factors affect are predicted, by the program code, to contribute to the behavior(s).

In some embodiments of the present invention, the program code determines that the user is exhibiting one or more behaviors indicative of a sensory issue simultaneously with determining context and eliminating or weighting behavior instances rendered unreliable, based on environmental factors likely impacting the behaviors and/or being the source of the behaviors. When data obtained by the program code indicates sensory issues, but the behaviors that predicate this indication are based on environmental factors, at least in part, rather than on sensory issues, determining that an issue exists would be a false positive. Environmental factors that represent a context in which behaviors that would indicate sensory issues in the absence of these factors include, but are not limited to, outside activities being performed by the user at the same time as the behaviors (the other activities could distract the user), degraded quality of input (e.g., too small font, lack of clarity, volume too low, soft enunciation, etc.), excessive noise, excessive light, and/or diminished light.

In some embodiments of the present invention, in generating and training the model (320), the program code continuously monitors the individual data and external data affecting the individual and adjusts the model, as well as the predicted sensory issues, based on dynamic changes obtained via the monitoring. In embodiments of the present invention, when the program code obtains data from sensors, in real-time, which conflicts with modeled patterns, the program code can either override the predictions of the model and/or update the model to comport with the anomalies.

In some embodiments of the present invention, based on determining the probability that the user is experiencing the sensory issue, the program code identifies one or more actions to mitigate the sensory issue (380). In some embodiments of the present invention, the program code selects one or more actions from a pre-defined list of actions, based on the sensory issue. In some embodiments of the present invention, the action taken by the program code is based on the observed (analyzed) severity of the identified sensory issue. For example, in some embodiments of the present invention, the program code determines that the behavioral patterns of a user, that indicate an issue, cross a given threshold. For example, after meeting a threshold for action, the program code can separate probabilities above the initial threshold into more and less severe indicators of the sensory issue, based on the size of the probability. Thus, based on a threshold being exceeded that indicated a higher probability, the program code notifies the user and/or a healthcare provider or proxy (with the permission of the user) of the issue. In some embodiments of the present invention, the program code determines that a sensory issue exists, but the behavioral patterns do not this higher threshold for severity (based on frequency, intensity, etc.) and the action taken by the program code is adjusting controls on a personal computing device. For example, if the program code determines that the user is experiencing hearing issues, the program code automatically increases the volume of one or more computing devices of the user (as the user earlier registered his or her devices). Also, if the program code determines that the user is experiencing vision issues that do not meet the higher notification threshold, the program code automatically adjusts certain visual settings on the devices of the user (font size, color, style) to make the graphic user interfaces displayed on the devices easier to read. In some embodiments of the present invention, the program code transmits a message to the user to change his or her distance from a visual or audio source. Thus, based on identifying one or more actions to mitigate the sensory issue indicated by the exhibited one or more behaviors, the program code initiates the one or more actions (390).

Embodiments of the present invention include a computer-implemented method, a computer system, and a computer program product where program code executing on one or more processors obtains a request to be electronically monitored, from a user, via a computing resource, where the request comprises authorization to access one or more data sources utilized by the user or proximate to the user. The program code continuously monitors the authorized one or more data sources to obtain data relevant to the user. The program code generates and trains a predictive model, where the predictive model is utilized by the one or more processors, to determine a probability that the user is experiencing a sensory issue, based on the continuously monitoring, and obtaining additional data, via an Internet connection, from one or more computing resources communicatively coupled to the one or more processors, where the additional data comprises one or more behaviors indicating the sensory issue and one or more contextual factors that contribute to the one or more behaviors, where the data relevant to the user is utilized by the one or more processors to establish ranges of expected behaviors for the user, when the user is engaged in specific activities, where the predictive model comprises the expected behaviors for the user. The program code cognitively analyzes based on applying the predictive model, a portion of the data obtained by the continuously monitoring during a given time period, to determine that a user is exhibiting the one or more behaviors indicative of the sensory issue, where the one or more behaviors represent deviations, during the given time period, from one or more of the established ranges of expected behaviors for the user.

In some embodiments of the present invention, based on determining that the user is exhibiting the one or more behaviors indicative of a sensory issue during the given time period, the program code determines a context for each incidence of the one or more behaviors indicative of the sensory issue during the given time period. The program code adjusts, in the portion of the continuously obtained data, a portion of the instances where the context comprises one or more of the one or more contextual factors, to generate an adjusted portion of the continuously obtained data.

In some embodiments of the present invention, the program code cognitively analyzes utilizing the predictive model, the adjusted portion, to determine the probability that the user is experiencing the sensory issue during the given time period.

In some embodiments of the present invention, where the adjusting by the program code comprises: for each instance of the one or more behaviors in the portion of the continuously obtained data: the program code determines whether the context includes at least one contextual factor of the one or more contextual factors. The program code determines, based on applying the predictive model, a probability that the at least one factor contributes to the one or more behaviors in the instance. The program code includes the instance in the portion of the instances based on the probability that the at least one factor contributes to the one or more behaviors in the instance exceeding a pre-defined threshold.

In some embodiments of the present invention, the program code, based on determining the probability that the user is experiencing the sensory issue during the given time period, identifies one or more actions to mitigate the sensory issue. The program code initiates the one or more actions. In some embodiments of the present invention, the one or more actions are identified based on a value of the probability, a first action comprises the one or more actions if the probability exceeds a pre-defined threshold, and a second action comprises the one or more actions if the probability is less than or equal to the pre-defined threshold. In some embodiments of the present invention, the first action comprises transmitting a notification to the user, and the second action comprises automatically adjusting a setting of the computing resource. In some embodiments of the present invention, the one or more actions comprise the program code automatically adjusting one or more settings on a device selected from the group consisting of: the computing resource and a data source of the one or more data sources. In some embodiments of the present invention, the sensory issue comprises an issue pertaining to vision of the user, and the program code automatically adjusting the one or more settings comprises making a change to the device selected from the group consisting of: changing the font displayed in a graphical user interface displayed on the device, increasing the a size of the font displayed in the graphical user interface displayed on the device, changing a color contrast in the graphical user interface displayed on the device, changing a color of at least one object displayed in the graphical user interface displayed on the device, changing a resolution setting of the device, and changing a magnification setting of the device. In some embodiments of the present invention, the sensory issue comprises an issue pertaining to hearing of the user, user, and the program code automatically adjusts the one or more settings comprises making changing a volume setting of the device.

In some embodiments of the present invention, the one or more data sources comprise sensors and a portion of the sensors comprise Internet of Things devices.

In some embodiments of the present invention, the data sources comprise Internet of Things devices accessible to the public, and the portion of the data comprises data obtained from the Internet of Things devices accessible to the public.

In some embodiments of the present invention, the sensory issue comprises an issue pertaining to vision of the user, and the one or more behaviors are selected from the group consisting of: squinting, removing glasses, rubbing eyes, and positioning close to displayed text or images.

In some embodiments of the present invention, the sensory issue comprises an issue pertaining to hearing of the user, and the one or more behaviors are selected from the group consisting of: requesting repetition of audio, responding incorrectly to an audio prompt, and orienting a computing device at a progressively further distance from the user.

Referring now to FIG. 4, a schematic of an example of a computing node, which can be a cloud computing node 10. Cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove. In an embodiment of the present invention computing resource(s) 120 (FIG. 1), computing resource(s) 134 (FIG. 1), data source(s) 173 (FIG. 1), existing database 191 (FIG. 1), and general medical data 141 (FIG. 1) can each be understood as a cloud computing node 10 (FIG. 4) and if not a cloud computing node 10, then one or more general computing nodes that include aspects of the cloud computing node 10. Various examples of these resources may, together, comprise a hybrid cloud.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 4, computer system/server 12 that can be utilized as cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples include, but are not limited to: microcode, device drivers, redundant processing units, external disk drive arrays, RAID systems, tape drives, and data archival storage systems, etc.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs). Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter). Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

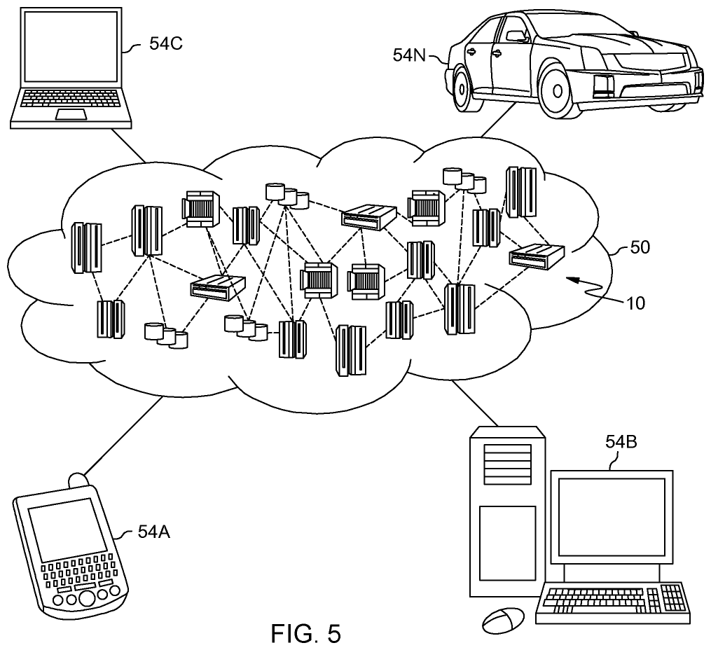

Referring now to FIG. 5, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 5 are intended to be illustrative only and that computing nodes 10 and cloud computing environment 50 can communicate with any type of computerized device over any type of network and/or network addressable connection (e.g., using a web browser).

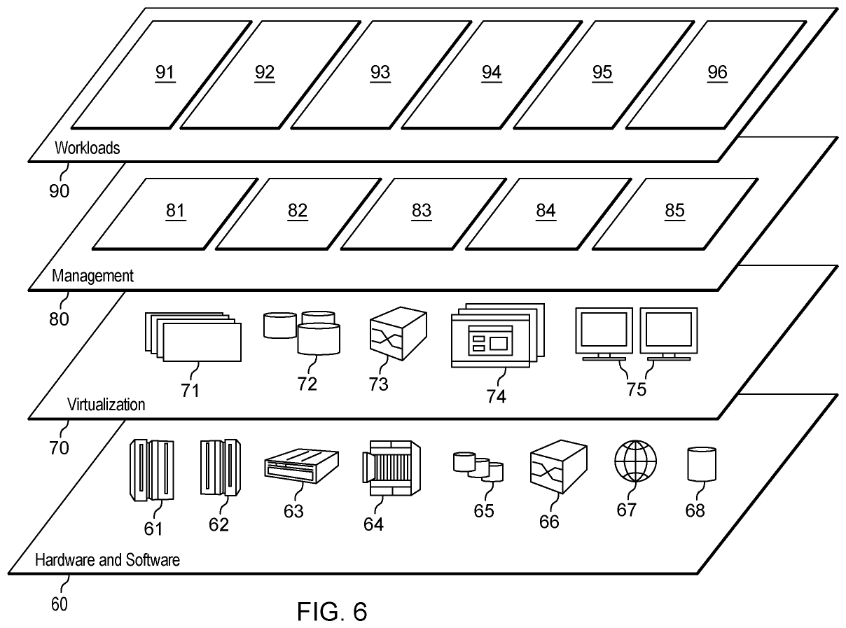

Referring now to FIG. 6, a set of functional abstraction layers provided by cloud computing environment 50 (FIG. 5) is shown. It should be understood in advance that the components, layers, and functions shown in FIG. 6 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and determining whether a user is experiencing a sensory issue (hearing, vision, etc.) 96.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer-implemented method, comprising:
obtaining, by one or more processors, a request to be electronically monitored, from a user, via a computing resource, wherein the request comprises authorization to access one or more data sources utilized by the user or proximate to the user;
continuously monitoring, by the one or more processors, the authorized one or more data sources to obtain data relevant to the user;
generating and training, by the one or more processors, a predictive model, wherein the predictive model is utilized by the one or more processors, to determine a probability that the user is experiencing a sensory issue, based on the continuously monitoring, and obtaining additional data, via an Internet connection, from one or more computing resources communicatively coupled to the one or more processors, wherein the additional data comprises one or more behaviors indicating the sensory issue and one or more contextual factors that contribute to the one or more behaviors, wherein the data relevant to the user is utilized by the one or more processors to establish ranges of expected behaviors for the user, when the user is engaged in specific activities, wherein the predictive model comprises the expected behaviors for the user; and
cognitively analyzing, by the one or more processors, based on applying the predictive model, a portion of the data obtained by the continuously monitoring during a given time period, to determine that a user is exhibiting the one or more behaviors indicative of the sensory issue, wherein the one or more behaviors represent deviations, during the given time period, from one or more of the established ranges of expected behaviors for the user.

2. The computer-implemented method of claim 1, further comprising:
based on determining that the user is exhibiting the one or more behaviors indicative of a sensory issue during the given time period, determining, by the one or more processors, a context for each incidence of the one or more behaviors indicative of the sensory issue during the given time period; and
adjusting, by the one or more processors, in the portion of the continuously obtained data, a portion of the instances where the context comprises one or more of the one or more contextual factors, to generate an adjusted portion of the continuously obtained data. and

[symbol]

3. The computer-implemented method of claim 2, further comprising:
cognitively analyzing, by the one or more processors, utilizing the predictive model, the adjusted portion, to determine the probability that the user is experiencing the sensory issue during the given time period.

4. The computer-implemented method of claim 2, wherein the adjusting comprises:
for each instance of the one or more behaviors in the portion of the continuously obtained data:
determining, by the one or more processors, whether the context includes at least one contextual factor of the one or more contextual factors;
determining, by the one or more processors, based on applying the predictive model, a probability that the at least one factor contributes to the one or more behaviors in the instance; and
including, by the one or more processors, the instance in the portion of the instances based on the probability that the at least one factor contributes to the one or more behaviors in the instance exceeding a pre-defined threshold.

5. The computer-implemented method of claim 3, further comprising:
based on determining the probability that the user is experiencing the sensory issue during the given time period, identifying, by the one or more processors, one or more actions to mitigate the sensory issue; and
initiating, by the one or more processors, the one or more actions.

6. The computer-implemented method of claim 5, wherein the one or more actions are identified based on a value of the probability, wherein a first action comprises the one or more actions if the probability exceeds a pre-defined threshold, and wherein a second action comprises the one or more actions if the probability is less than or equal to the pre-defined threshold.

7. The computer-implemented method of claim 6, wherein the first action comprises transmitting, by the one or more processors, a notification to the user, and wherein the second action comprises automatically adjusting, by the one or more processors, a setting of the computing resource.

8. The computer-implemented of claim 5, wherein the one or more actions comprise automatically adjusting one or more settings on a device selected from the group consisting of: the computing resource and a data source of the one or more data sources.

9. The computer-implemented method of claim 8, wherein the sensory issue comprises an issue pertaining to vision of the user, and wherein automatically adjusting the one or more settings comprises making a change to the device selected from the group consisting of: changing the font displayed in a graphical user interface displayed on the device, increasing the a size of the font displayed in the graphical user interface displayed on the device, changing a color contrast in the graphical user interface displayed on the device, changing a color of at least one object displayed in the graphical user interface displayed on the device, changing a resolution setting of the device, and changing a magnification setting of the device.

10. The computer-implemented method of claim 8, wherein the sensory issue comprises an issue pertaining to hearing of the user, user, and wherein automatically adjusting the one or more settings comprises making changing a volume setting of the device.

11. The computer-implemented method of claim 1, wherein the one or more data sources comprise sensors and a portion of the sensors comprise Internet of Things devices.

12. The computer-implemented method of claim 1, wherein the one or more data sources comprise Internet of Things devices accessible to the public, and wherein the portion of the data comprises data obtained, by the one or more processors, from the Internet of Things devices accessible to the public.

13. The computer-implemented method of claim 1, wherein the sensory issue comprises an issue pertaining to vision of the user, and the one or more behaviors are selected from the group consisting of: squinting, removing glasses, rubbing eyes, and positioning close to displayed text or images.

14. The computer-implemented method of claim 1, wherein the sensory issue comprises an issue pertaining to hearing of the user, and the one or more behaviors are selected from the group consisting of: requesting repetition of audio, responding incorrectly to an audio prompt, and orienting a computing device at a progressively further distance from the user.

15. A computer program product comprising:
a computer readable storage medium readable by one or more processors and storing instructions for execution by the one or more processors for performing a method comprising:
obtaining, by the one or more processors, a request to be electronically monitored, from a user, via a computing resource, wherein the request comprises authorization to access one or more data sources utilized by the user or proximate to the user;
continuously monitoring, by the one or more processors, the authorized one or more data sources to obtain data relevant to the user;
generating and training, by the one or more processors, a predictive model, wherein the predictive model is utilized by the one or more processors, to determine a probability that the user is experiencing a sensory issue, based on the continuously monitoring, and obtaining additional data, via an Internet connection, from one or more computing resources communicatively coupled to the one or more processors, wherein the additional data comprises one or more behaviors indicating the sensory issue and one or more contextual factors that contribute to the one or more behaviors, wherein the data relevant to the user is utilized by the one or more processors to establish ranges of expected behaviors for the user, when the user is engaged in specific activities, wherein the predictive model comprises the expected behaviors for the user; and
cognitively analyzing, by the one or more processors, based on applying the predictive model, a portion of the data obtained by the continuously monitoring during a given time period, to determine that a user is exhibiting the one or more behaviors indicative of the sensory issue, wherein the one or more behaviors represent deviations, during the given time period, from one or more of the established ranges of expected behaviors for the user.

16. The computer program product of claim 15, the method further comprising:based on determining that the user is exhibiting the one or more behaviors indicative of a sensory issue during the given time period, determining, by the one or more processors, a context for each incidence of the one or more behaviors indicative of the sensory issue during the given time period; and
adjusting, by the one or more processors, in the portion of the continuously obtained data, a portion of the instances where the context comprises one or more of the one or more contextual factors, to generate an adjusted portion of the continuously obtained data.

17. The computer program product of claim 16, the method further comprising:
cognitively analyzing, by the one or more processors, utilizing the predictive model, the adjusted portion, to determine the probability that the user is experiencing the sensory issue during the given time period.

18. The computer program product of claim 16, wherein the adjusting comprises:
for each instance of the one or more behaviors in the portion of the continuously obtained data:
determining, by the one or more processors, whether the context includes at least one contextual factor of the one or more contextual factors;
determining, by the one or more processors, based on applying the predictive model, a probability that the at least one factor contributes to the one or more behaviors in the instance; and
including, by the one or more processors, the instance in the portion of the instances based on the probability that the at least one factor contributes to the one or more behaviors in the instance exceeding a pre-defined threshold.

19. The computer program product of claim 17, the method further comprising:
based on determining the probability that the user is experiencing the sensory issue during the given time period, identifying, by the one or more processors, one or more actions to mitigate the sensory issue; and
initiating, by the one or more processors, the one or more actions.

20. A system comprising:
a memory;
one or more processors in communication with the memory;
program instructions executable by the one or more processors via the memory to perform a method, the method comprising:
obtaining, by the one or more processors, a request to be electronically monitored, from a user, via a computing resource, wherein the request comprises authorization to access one or more data sources utilized by the user or proximate to the user;
continuously monitoring, by the one or more processors, the authorized one or more data sources to obtain data relevant to the user;
generating and training, by the one or more processors, a predictive model, wherein the predictive model is utilized by the one or more processors, to determine a probability that the user is experiencing a sensory issue, based on the continuously monitoring, and obtaining additional data, via an Internet connection, from one or more computing resources communicatively coupled to the one or more processors, wherein the additional data comprises one or more behaviors indicating the sensory issue and one or more contextual factors that contribute to the one or more behaviors, wherein the data relevant to the user is utilized by the one or more processors to establish ranges of expected behaviors for the user, when the user is engaged in specific activities, wherein the predictive model comprises the expected behaviors for the user; and
cognitively analyzing, by the one or more processors, based on applying the predictive model, a portion of the data obtained by the continuously monitoring during a given time period, to determine that a user is exhibiting the one or more behaviors indicative of the sensory issue, wherein the one or more behaviors represent deviations, during the given time period, from one or more of the established ranges of expected behaviors for the user.
